(19) United States
(12) Patent Application Publication
(10) Pub. No.: US 2019/0139643 A1
(43) Pub. Date: May 9, 2019

(54) FACILITATING MEDICAL DIAGNOSTICS WITH A PREDICTION MODEL

(71) Applicant: International Business Machines Corporation, Armonk, NY (US)

(72) Inventors: Xiu Li Li, Beijing (CN); Guo Tong Xie, Xi Er Qi (CN); Xiaolu Zhang, Beijing (CN); Shi Wan Zhao, Beijing (CN)

(21) Appl. No.: 15/806,827

(22) Filed: Nov. 8, 2017

(51) Int. Cl. G16H 50/20; G16H 10/60; G06N 5/02

(52) U.S. Cl. CPC G16H 50/20; G06N 5/02; G16H 10/60

(57) ABSTRACT

Techniques that facilitate improved medical condition diagnostics are provided. An example embodiment can include a device. The device can include a memory that stores computer executable components and a processor. The processor can execute the computer executable components stored in the memory. The computer executable components can include training logic component and a determination logic component. The training logic component can generate a prediction model. The prediction model can generate \predict diagnosis based on electronic healthcare record data and image data of a known patient. The determination logic component can determine whether the predicted diagnosis exceeds an accuracy threshold value.

### BACKGROUND

The subject disclosure relates to prediction models, and more specifically, to prediction models to improve medical diagnostics.

### SUMMARY

The following presents a summary to provide a basic understanding of one or more embodiments of the invention. This summary is not intended to identify key or critical elements, or delineate any scope of the particular embodiments or any scope of the claims. Its sole purpose is to present concepts in a simplified form as a prelude to the more detailed description that is presented later. In one or more embodiments described herein, devices, systems, computer-implemented methods, apparatus, and/or computer program products that facilitate medical diagnostics by creating a prediction model to diagnose future patients.

An example embodiment, can include a device. The device can include a memory that stores computer executable components and a processor. The processor can execute the computer executable components stored in the memory. The computer executable components can include training logic component and a determination logic component. The training logic component can generate a prediction model. The prediction model can generate a diagnosis of a medical patient based on electronic healthcare record data and image data of a known patient. The determination logic component can determine whether the predicted diagnosis exceeds an accuracy threshold value.

Another embodiment, can include a computer-implemented method. The method can generate, by a system operatively coupled to a processor, a prediction model. The prediction model can generate, by the system, a predicted diagnosis based on electronic healthcare record data and image data of a known patient. The method can determine, by the system, whether the predicted diagnosis exceeds an accuracy threshold value.

Other embodiments may include a computer program product that facilitates medical diagnosis. The computer program product can include a computer readable storage medium having program instructions embodied therewith. The program instructions can be executable by a processor to generate, by the processor, a prediction model. The prediction model generates a predicted diagnosis based on electronic healthcare record data and image data of a known patient. The program instructions can be executable by a processor to determine, by the processor, whether the predicted diagnosis exceeds an accuracy threshold value.

### DESCRIPTION OF THE DRAWINGS

FIG. 1 illustrates a block diagram of an example, non-limiting block diagram of a system and environment for creating a prediction model that facilitates better diagnose medical conditions in accordance with one or more embodiments described herein.

FIG. 2 illustrates a block diagram of an example, non-limiting device for creating a prediction model that facilitates better diagnoses of medical conditions in accordance with one or more embodiments described herein.

FIG. 3 illustrates another block diagram of an example, non-limiting device for creating a prediction model that facilitates better diagnoses of medical conditions in accordance with one or more embodiments described herein.

FIG. 4 illustrates another block diagram of an example, non-limiting device for creating a prediction model that facilitates better diagnoses of medical conditions in accordance with one or more embodiments described herein.

FIG. 5 illustrates another block diagram of an example, non-limiting device for creating a prediction model that facilitates better diagnoses of medical conditions in accordance with one or more embodiments described herein.

FIG. 6 illustrates a block diagram of an example, non-limiting block diagram of a system and environment for creating a prediction model that facilitates better diagnoses of medical conditions in accordance with one or more embodiments described herein.

FIG. 7 illustrates a flow diagram of an example, non-limiting computer-implemented method that facilitates generating a prediction model in accordance with one or more embodiments described herein.

FIG. 8 illustrates a block diagram of an example; non-limiting operating environment in which one or more embodiments described herein can be facilitated.

FIG. 9 illustrates an example, non-limiting; networking environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

The various embodiments and configurations of the invention described herein can relate to improving diagnosis of medical diseases. The various embodiments and configurations can be useful in clinical situations where “clinical” refers to the observation and/or treatment of actual patients out in the “field” rather than theoretical hospital studies or laboratory research. Initially, a prediction model can be created at a hospital or medical research facility using large amounts of known/prior medical data of known patients and their outcomes. As discussed in more detail below, this model can be provided to devices used by doctors in the field so those clinical doctors can make a better diagnosis in the clinical setting where the clinical doctor has limited information based essentially on his current patient. For example, the prediction model can be run on a local device by the clinical doctor with his patient's current medical data to predict a diagnosis of that patient using that patient's limited clinical data in combination with the prediction model that is based on much more prior patient data. This approach likely leads to a much accurate diagnosis of the current patient.

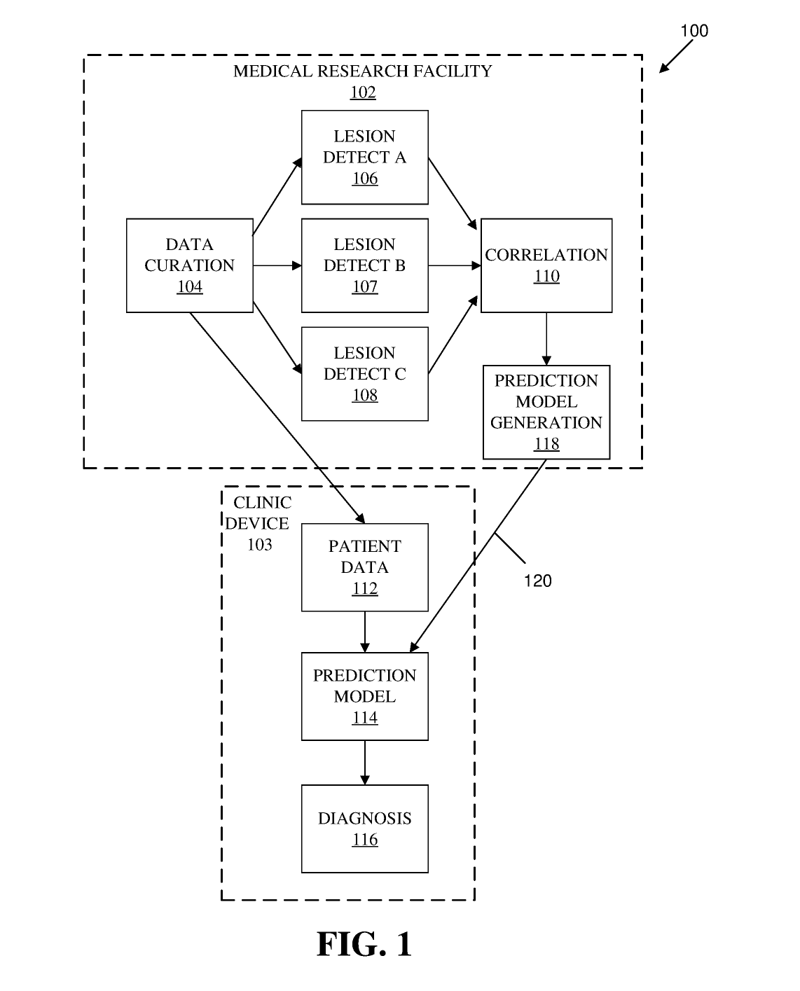

FIG. 1 illustrates an example, non-limiting environment 100 where a medical research facility 102 can generate a prediction model 114 that is later used in a clinical environment where a clinical device 103 can be located. In general, the prediction model 114 can be generated by schematic blocks representing stages and actions used to produce the prediction model 114 at the medical research facility 102. The blocks can include a data curation block 104, a first lesion block 106, a second lesion block 107, a third lesion block 108, a correlation block 110, and a prediction model generation block 118. The data curation block 104 can collect/receive input data, and then can format, organize, store, and process the input data in other ways. This processed data can then be passed to each of the lesion blocks. The first lesion block 106 can detect type A lesions in the data, the second lesion block 107 can detect type B lesions in the data, and the third lesion block 108 can detect type C lesions in the data. The lesion blocks output data can then be correlated in the correlation block 110 and the resulting correlation can be input to the prediction model generation block 118. The prediction model generation block 118 can then generate the prediction model 114 based partly on the correlation results and can then send via line 120 the prediction model 114 to the clinical device 103 where it can be used to diagnose new and different patients.

The clinical device 103 can be locate at a clinical office remote from where the medical research facility 102 where the prediction model 114 was produced. The clinical device 103 is represented by a patient data block 112, a diagnosis block 116, and a block representing the prediction model 114. An example diagnosis by the clinical device 103 at a remote clinic can be performed by receiving and processing a new patient's data at the patient data block 112. This data can then be processed by the block representing the prediction model 114. The resulting diagnosis can then be generated, formatted, and/or output or displayed from the diagnosis block 116.

Thus, an environment similar to the environment of FIG. 1 can use endoscopy images and other multi-modality data of many patients to train a digestive diseases prediction model. Based on this model, a device using the digestive diseases prediction model can help clinical doctors screen digestive diseases at an early stage.

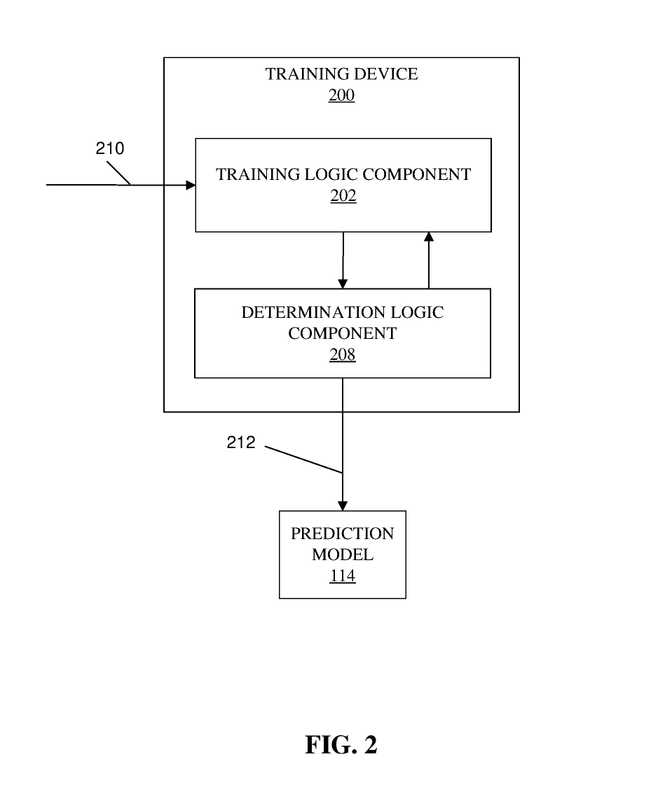

FIG. 2 illustrates in more detail a block diagram of an example, non-limiting embodiment of a training device 200 that creates prediction models for use in diagnosing medical conditions. Repetitive description of like elements employed in other embodiments described herein is omitted for the sake of brevity.

In various embodiments, the training device 200 can include a training logic component 202 and a determination logic component 208. An input line 210 can contain one or more communication lines that provide medical data associated with a patient into the training device 200. An output line 212 can contain one or more individual output lines to bring signal(s) specifying the prediction model 114 created by the training logic component 202 that can be used by other devices to predict future medical conditions. The input line 210 and the output line 212 can be single lines or multiple lines/busses and can be part of the training device 200 or can be communicatively, removably attached to the training device 200 (or device ports).

The training device 200 can be employed to use hardware and/or software to solve problems be encounter when medical diagnosis predictions and that can be highly technical in nature, that are not abstract and that cannot be performed as a set of mental acts by a human A human cannot use mental acts to produce a timely and useful medical diagnosis prediction model that can be used to analyze new and different patients. This is because the vast amount of data required to generate and produce a general prediction model that can be used on many different new patients would require hundreds of thousands, or millions, or even more data from even more past patient cases, the more data the better the model. A robust prediction model developed with this vast amount of data cannot be timely comprehended and processed, nor processed in parallel way by a human. Further, some of the processes performed by the training device 200 can in other embodiments, be performed by a processor (or computer) carrying out defined tasks related to memory operations. In summary, A human would take many years or lifetimes to process vast amounts of medical data including making comparisons between similar data values after finding those values for each of millions of past medical cases, sorting the data, and performing other calculations with that data to produce a useful prediction model.

“Processor”, “Logic”, and “Logic Component” as used herein, includes but is not limited to hardware, firmware, software and/or combinations of each to perform a function(s) or an action(s), and/or to cause a function or action from another logic, method, and/or system. For example, based on a desired application or need, logic and/or processor can include a software-controlled microprocessor, discreet logic, an application specific integrated circuit (ASIC), a programmed logic device, a memory device containing instructions or the like. Logic, logic component, and/or processor can include one or more gates, combinations of gates, or other circuit components. Logic, logic component, and/or processor can also be fully embodied as software. Where multiple logics and/or processors are described, it can be possible to incorporate the multiple logics and/or processors into one physical logic (or processor or logic component). Similarly, where a single logic, logic component, and/or processor is described, it can be possible to distribute that single logic, logic component, and/or processor between multiple physical logics, logic components, and/or processors.

The training logic component 202 can use electronic healthcare record (EHR) data (EHR data) and image data of known patients to generate the prediction model 114. For example, the training logic component 202 can generate prediction model can be generated by a processor processing patient data making many simple comparisons between vast amounts of data from different patients. As understood by those of ordinary skill in the art, these comparisons may be correlated by a mathematical equation or in another way. These correlation results may then be analyzed and processed by computer to generate a prediction model 114 that is based on a vast number of know patients and their known diagnostic results.

The prediction model 114 can later generate a predicted diagnosis of at least one medical condition of a new patient associated with new EHR data and new image data of the new patient. For example, the prediction model 114 receive input data representing the new patient's EHR data and image data. This data can then be processed by a complex mathematical formula or with a series of mathematical steps to correlate or compare the new patient's data with one or more algorithms within the prediction model 114 that were generated earlier based on a vast number of prior patients.

The determination logic component 208 can determine if the predicted diagnosis exceeds an accuracy threshold value. In other embodiments, the determination logic component 208 can determine if the predicted diagnosis crosses the accuracy threshold value. The accuracy threshold value can be a value representing a minimum accuracy of prediction that is acceptable for the prediction model 114.

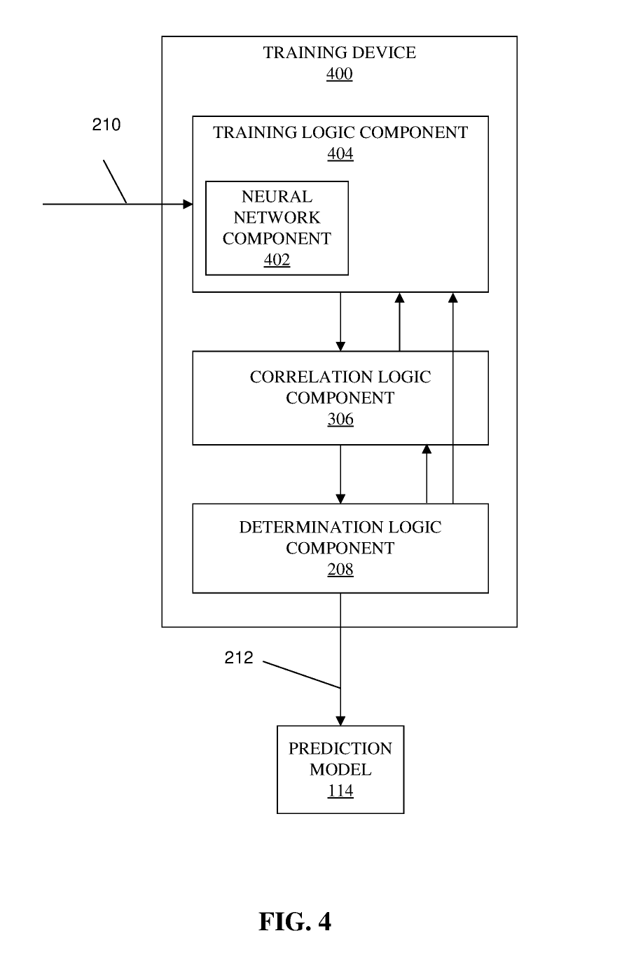

If the predicted diagnosis does not exceed the accuracy threshold value, the training logic component 202 can revise the prediction model 114 based on the comparison results and feedback from the determination logic component 208. For example, a learning algorithm may be used to update the prediction model when the prediction model is based on neural networks, as understood by those of ordinary skill in the art and as discussed in more detail below and with reference to FIG. 4.

On a repeated attempt to generate a prediction model 114 that is an improved prediction model, the determination logic component 208 can detect that the predicted diagnosis has then exceeded (or crossed) the accuracy threshold value. If not, the determination logic component 208 feeds its results back to the training logic component 202 so that a new prediction model can be generated based on the fed back information. Once the predicted diagnosis crosses the accuracy threshold value and is accepted for the different patients represented by the EHR data and the image data, the prediction model 114 can be considered as completed and usable by other medical devices at clinical offices.

In more detail, an Electronic Health Record (EHR) can be an electronic version of a patient's medical history. The EHR is maintained by a provider over time, and can include much and sometimes all the key administrative clinical data relevant to that persons care under a particular provider, including demographics, progress notes, problems, medications, and the like. Essentially, an electronic health record (EHR) is a digital version of a patient's paper chart. EHRs are real-time, patient-centered records that can make information available instantly and securely to authorized users. While an EHR can contain the medical and treatment histories of patients, an EHR system can be built to go beyond standard clinical data collected in a provider's office and can be inclusive of a broader view of a patient's care.

For example, EHRs can contain a patient's medical history, diagnoses, medications, treatment plans, immunization dates, allergies, radiology images, and laboratory and test results. EHRs can allow access to evidence-based tools that providers can use to make decisions about a patient's care EHRs can also automate and streamline provider workflow.

When in an electronic format, EHRs can provide for health information that can be created and managed by authorized providers in a digital format capable of being shared with other providers across more than one health care organization. EHRs can be built to share information with other health care providers and organizations—such as laboratories, specialists, medical imaging facilities, pharmacies, emergency facilities, and school and workplace clinics, and the like.

In some embodiments, the prediction model 114 can identify diagnose digestive track conditions. As previously mentioned, digestive track conditions are hard to diagnose unless a doctor can look over a long timeframe of conditions, which may often not be possible. The EHR data and other images spanning many years for other patients make it possible to create a prediction model 114 that accomplishes that. In one configuration of this embodiment, the image data and the prediction model can be used by the prediction model 114 and the determination logic component 208 to indicate if there are polyps, bleeding, and/or an ulcer are associated with a lesion.

In other embodiments, the training logic component 202 can generate the prediction model 114 to exceed the accuracy threshold value using a learning algorithm. In at least one configuration, the training logic component 202 can apply assisted learning techniques to the learning algorithm. In other implementations, the training logic component 202 can also improve an accuracy of the prediction model 114 by applying a directed learning algorithm to the prediction model 114. The prediction model 114 can further have one or more weighted values. Some of the weighted values are modified when the directed learning algorithm applies known values to the prediction model 114 and propagates signals through the prediction model 114. At least some of the weighted values can be modified based, at least in part, on the signals propagated through the prediction model 114. The weighted values can be modified based, at least in part, on the signals to improve the accuracy of the prediction model 114.

The training device 200 can be implemented with other useful features and components. For example, the EHR data can at least partially be comprised of different data collected at different times. The EHR data can include report data associated with medical consultation reports. Additionally, the prediction model 114 can have the ability to detect internal lesions, cancer, aneurisms, tumors, and/or arteriovasuclar malformations (AVMs).

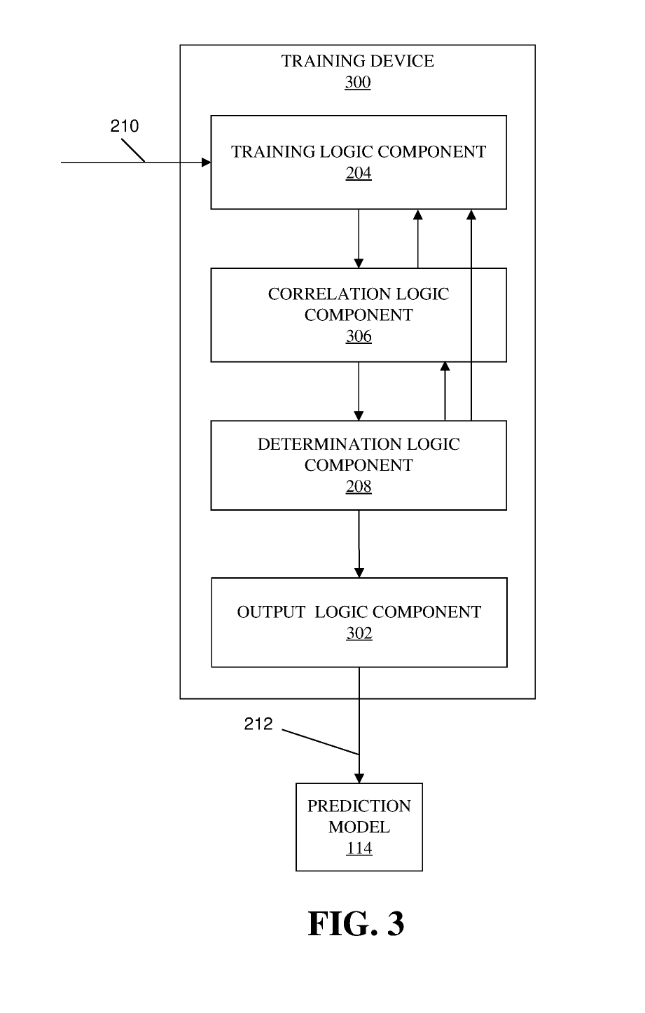

FIG. 3 illustrates another block diagram of an example, non-limiting embodiment of a training device 300 that creates prediction models for use in medical diagnosis. Repetitive description of like elements employed in other embodiments described herein is omitted for sake of brevity.

The training device 300 can include the training logic component 202, the determination logic component 208, the input line 210, and the output line 212 of the training device 200 of FIG. 2. The training device 300 of FIG. 3 can further include an output logic component 302 and a correlation logic component 306. The output logic component 302 can output the revised prediction model for use by other medical devices to generate a new medical diagnosis based on new image data of the new patient used to create the new prediction model. In some configurations, the output logic component 302 can include a transmitter that can transmit the completed prediction model either as wired or wireless data to one or more remote clinics.

The correlation logic component 306 can compare a known diagnosis with the predicted diagnosis and can, based on that comparison, generate comparison results. The determination logic component 208 can also determine if the comparison results exceed the accuracy threshold value. Responsive to the comparison results not exceeding the accuracy threshold value, the training logic component 202 can revise the prediction model based on the comparison results to produce a revised prediction model better at diagnosing an unknown medical condition of a new patient.

FIG. 4 illustrates another block diagram of an example, non-limiting embodiment of a training device 400 that creates prediction models for use in medical diagnosis. Repetitive description of like elements employed in other embodiments described herein is omitted for sake of brevity.

The training device 400 can include the correlation logic component 306, the determination logic component 208, the input line 210, and the output line 212 of the training device 200 of FIG. 2. A training logic component 404 of FIG. 4 can further include a neural network component 402 that can simulate neural networks. In some configurations, the neural network component 402 can model an array of artificial neurons having a front row of neurons and a back row of neurons. The training logic component 404 can apply known inputs to the front row of neurons causing a generation of signals within the array of artificial neurons. The training logic component 404 with the neural network component 402 can improve an accuracy of the prediction model by adjusting weight values indicating strength of connections between neurons of the array of artificial neurons. The weight values indicating strength of connections between neurons may be updated using any suitable learning algorithm as understood by those of ordinary skill in training neural network models.

For example, a backpropagation algorithm can train a multilayer neural network for a given set of input patterns with known classifications. When each entry of the sample set is presented to the network, the network examines its output response to the sample input pattern. The output response is then compared to the known and desired output and the error value is calculated. Based on the error, the connection weights between modeled neurons are adjusted. The backpropagation algorithm can be based on the Widrow-Hoff delta learning rule in which the weight adjustment can be performed through a mean square error of the output response to the sample input. The set of these sample patterns can be repeatedly presented to the network until the error value is minimized

In additional embodiments, the neural network component 402 can utilize various neural networks in other ways. In general, a patient's current image data can be used for diagnosis; however, one-time snapshot image data is static and hard to use for prognosis due to the lack of trending information. On the other hand, temporal EHR data usually has long duration and can be used to discover trends of the disease progression. Thus, some embodiments can link EHR data with image data. For EHR data and image data aligned at a similar time point, the neural network component 402 can implement a recurrent neural network (RNN) to construct an EHR feature vector, which represents the patient's status until a time point. In addition, a convolution neural network (CNN) can be leveraged to construct an image feature vector, and then can learn a generator model, which takes the EHR feature vector as input and generates the image feature vector. With this learned generator model, given a new and different patient's EHR data, for example in a clinic environment. This configuration of the neural network component 402 can generate a sequence of image vectors and then can use a sequence of image vectors to predict a cancer probability or another probability.

The mechanism by which the CCN and RNN represent these patterns can be different. In the case of a CNN, the neural network can be looking for the same patterns on different subfields of the image. In the case of a RNN, the neural network can be (in a simple case) feeding the hidden layers from the previous step as an additional input into the next step. While the RNN builds up memory in this process, it is not looking for the same patterns over different slices of time in the same way that a CNN is looking for the same patterns over different regions of space.

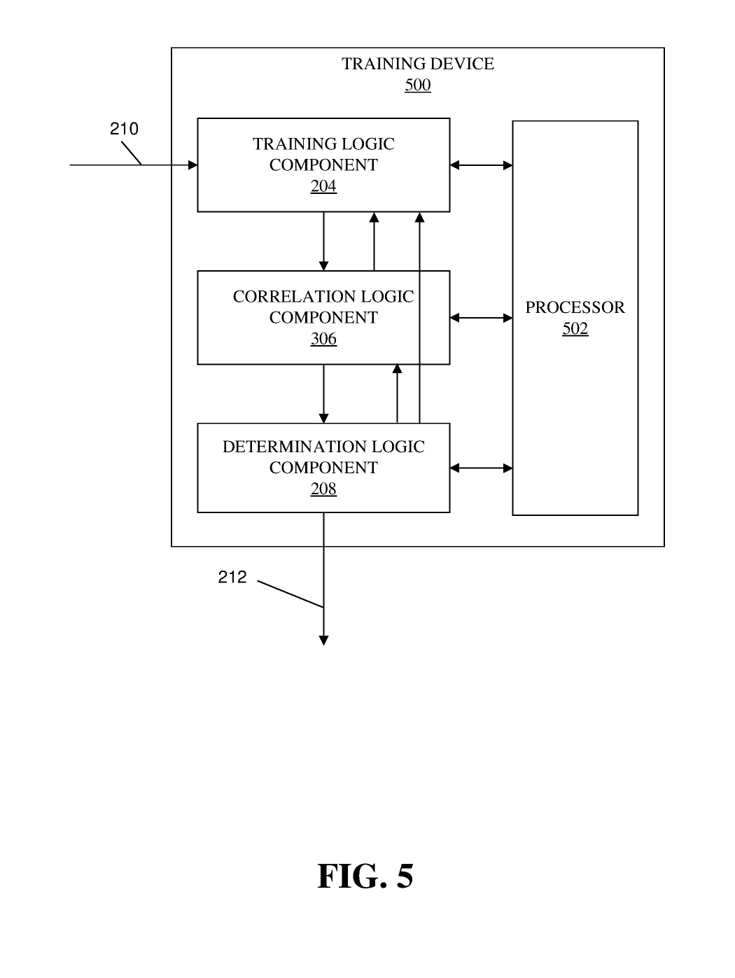

FIG. 5 illustrates another block diagram of an example, non-limiting embodiment of a training device 500 that creates prediction models for use in medical diagnosis. Repetitive description of like elements employed in other embodiments described herein is omitted for sake of brevity.

The training device 500 can include the training logic component 202, the correlation logic component 306, the determination logic component 208, the input line 210, and the output line 212 of the training device 200 of FIGS. 2 and 3. The training device 500 of FIG. 5 can further include a processor 502 that can provide control and assistance to the training logic component 202, the correlation logic component 306, and/or the determination logic component 208. The processor 502 can assist components by executing algorithms, portions of algorithms, and the like for one or more of the components of FIG. 5 to ensure that one or more of the components does not become a bottleneck in the performance of the training device 500.

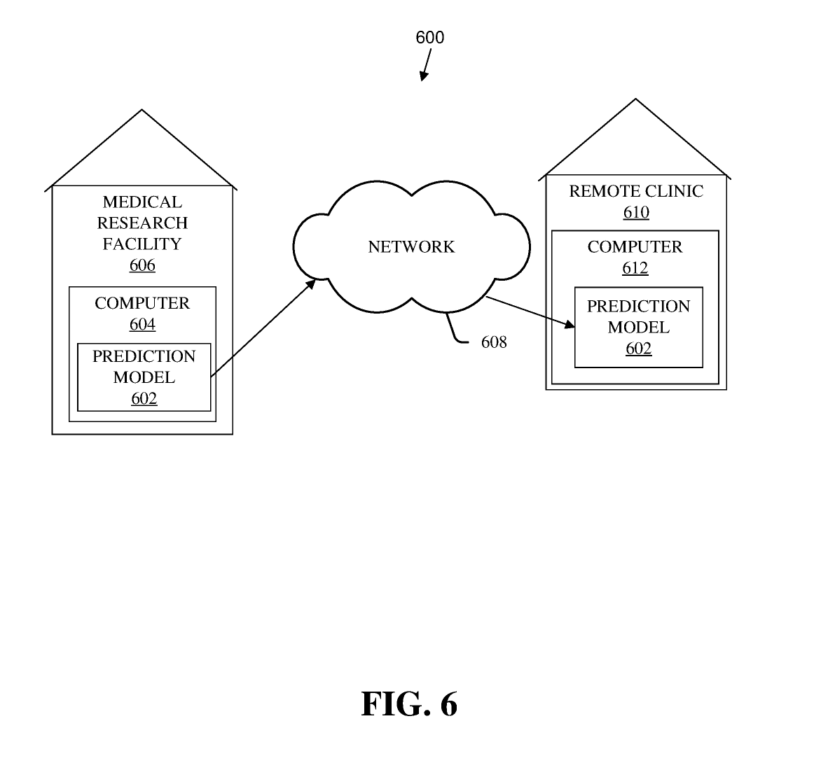

FIG. 6 illustrates another block diagram of an example, non-limiting embodiment of an environment 600 in which a prediction model 602 can operate. Repetitive description of like elements employed in other embodiments described herein is omitted for sake of brevity.

The prediction model 602 can be generated with a computer 604 or the like at a medical research facility 606 as discussed above.

The computer 604 can transmit the prediction model 602 over a network 608 wired and/or wireless to a remote clinic 610. The prediction model 602 can be received by a device or computer 612 at the remote clinic 610 and used later to diagnose clinical patients. For example, the prediction model 602 may be loaded into the computer 612 at the remote clinic 610 so that it may be used with local diagnostic software. When seeing a clinical patient, a doctor can input a new patient's medical symptoms into the diagnostic software (SW) and this SW can then process the patient's symptoms, electronic health record, and/or image data together with the SW utilizing the prediction model 602 to produce one or more possible causes/diagnoses of the patient's medical condition.

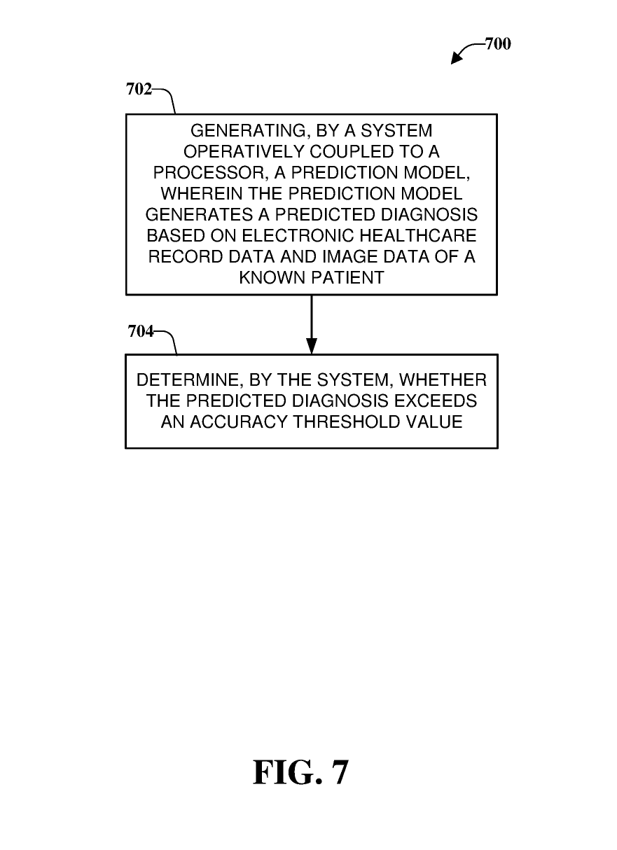

FIG. 7 illustrates a flow diagram of an example, non-limiting embodiment of a computer-implemented method 700 that can generate a prediction model. Repetitive description of like elements employed in other embodiments described herein is omitted for sake of brevity.

In one embodiment, the computer-implemented method 700 can begin, at 702, generating, by a system operatively coupled to a processor, at 702, a prediction model. The prediction model can generate a predicted diagnosis based on electronic healthcare record data and image data of a known patient. The method 700 determines, by the system, whether the predicted diagnosis exceeds an accuracy threshold value

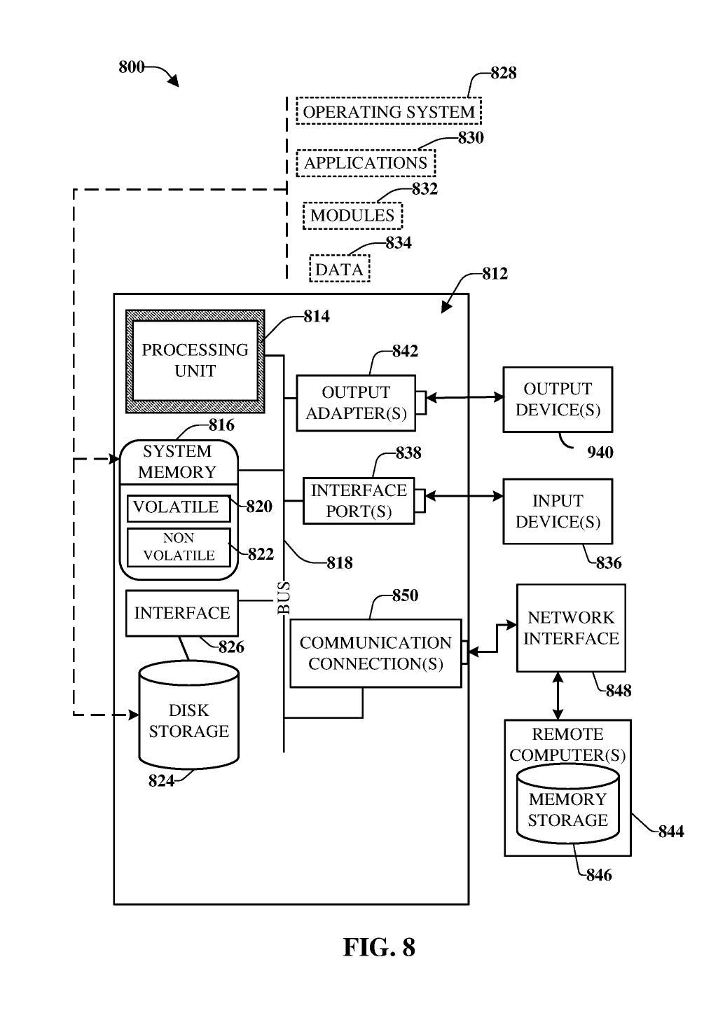

To provide a context for the various aspects of the disclosed subject matter, FIG. 8 as well as the following discussion are intended to provide a general description of a suitable environment in which the various aspects of the disclosed subject matter can be implemented. FIG. 8 illustrates a block diagram of an example; non-limiting operating environment in which one or more embodiments described herein can be facilitated. Repetitive description of like elements employed in other embodiments described herein is omitted for sake of brevity. With reference to FIG. 8, a suitable operating environment 800 for implementing various aspects of this disclosure can also include a computer 812. The computer 812 can also include a processing unit 814, a system memory 816, and a system bus 818. The system bus 818 couples system components including, but not limited to, the system memory 816 to the processing unit 814. The processing unit 814 can be any of various available processors. Dual microprocessors and other multiprocessor architectures also can be employed as the processing unit 814. The system bus 818 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Firewire (IEEE 1394), and Small Computer Systems Interface (SCSI).

The system memory 816 can also include volatile memory 820 and nonvolatile memory 822. The basic input/output system (BIOS), containing the basic routines to transfer information between elements within the computer 812, such as during start-up, is stored in nonvolatile memory 822. By way of illustration, and not limitation, nonvolatile memory 822 can include read only memory (ROM), programmable ROM (PROM), electrically programmable ROM (EPROM), electrically erasable programmable ROM (EEPROM), flash memory, or nonvolatile random access memory (RAM) (e.g., ferroelectric RAM (FeRAM). Volatile memory 820 can also include random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM.

Computer 812 can also include removable/non-removable, volatile/non-volatile computer storage media. FIG. 8 illustrates, for example, a disk storage 824. Disk storage 824 can also include, but is not limited to, devices like a magnetic disk drive, floppy disk drive, tape drive, Jaz drive, Zip drive, LS-100 drive, flash memory card, or memory stick. The disk storage 824 also can include storage media separately or in combination with other storage media including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 824 to the system bus 818, a removable or non-removable interface is typically used, such as interface 826. FIG. 8 also depicts software that acts as an intermediary between users and the basic computer resources described in the suitable operating environment 800. Such software can also include, for example, an operating system 828. Operating system 828, which can be stored on disk storage 824, acts to control and allocate resources of the computer 812.

System applications 830 take advantage of the management of resources by operating system 828 through program modules 832 and program data 834, e.g., stored either in system memory 816 or on disk storage 824. It is to be appreciated that this disclosure can be implemented with various operating systems or combinations of operating systems. A user enters commands or information into the computer 812 through input device(s) 836. Input devices 836 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 814 through the system bus 818 via interface port(s) 838. Interface port(s) 838 include, for example, a serial port, a parallel port, a game port, and a universal serial bus (USB). Output device(s) 840 use some of the same type of ports as input device(s) 836. Thus, for example, a USB port can be used to provide input to computer 812, and to output information from computer 812 to an output device 840. Output adapter 842 is provided to illustrate that there are some output devices 840 like monitors, speakers, and printers, among other output devices 840, which require special adapters. The output adapters 842 include, by way of illustration and not limitation, video and sound cards that provide a means of connection between the output device 840 and the system bus 818. It should be noted that other devices and/or systems of devices provide both input and output capabilities such as remote computer(s) 844.

Computer 812 can operate in a networked environment using logical connections to one or more remote computers, such as remote computer(s) 844. The remote computer(s) 844 can be a computer, a server, a router, a network PC, a workstation, a microprocessor based appliance, a peer device or other common network node and the like, and typically can also include many or all of the elements described relative to computer 812. For purposes of brevity, only a memory storage device 846 is illustrated with remote computer(s) 844. Remote computer(s) 844 is logically connected to computer 812 through a network interface 848 and then physically connected via communication connection 850. Network interface 848 encompasses wire and/or wireless communication networks such as local-area networks (LAN), wide-area networks (WAN), cellular networks, etc. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL). Communication connection(s) 850 refers to the hardware/software employed to connect the network interface 848 to the system bus 818. While communication connection 850 is shown for illustrative clarity inside computer 812, it can also be external to computer 812. The hardware/software for connection to the network interface 848 can also include, for exemplary purposes only, internal and external technologies such as, modems including regular telephone grade modems, cable modems and DSL modems, ISDN adapters, and Ethernet cards.

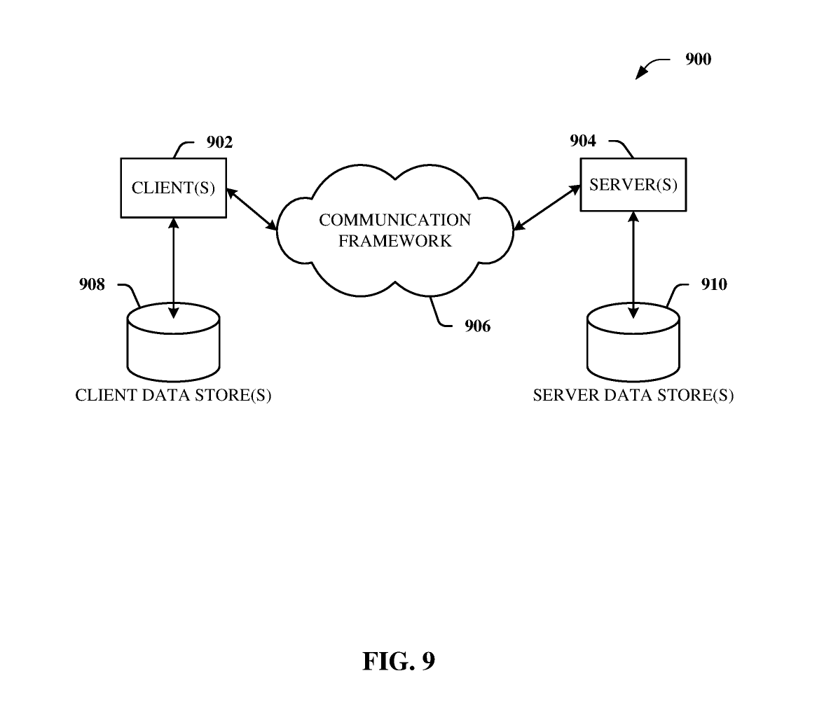

FIG. 9 is a schematic block diagram of a sample computing environment 900 with which the disclosed subject matter can interact. The sample computing environment 900 includes one or more client(s) 902. The client(s) 902 can be hardware and/or software (e.g., threads, processes, computing devices). The sample computing environment 900 also includes one or more server(s) 904. The server(s) 904 can also be hardware and/or software (e.g., threads, processes, computing devices). The servers 904 can house threads to perform transformations by employing one or more embodiments as described herein, for example. One possible communication between a client 902 and servers 904 can be in the form of a data packet adapted to be transmitted between two or more computer processes. The sample computing environment 900 includes a communication framework 906 that can be employed to facilitate communications between the client(s) 902 and the server(s) 904. The client(s) 902 are operably connected to one or more client data store(s) 908 that can be employed to store information local to the client(s) 902. Similarly, the server(s) 904 are operably connected to one or more server data store(s) 910 that can be employed to store information local to the servers 904.

The present invention may be a system, a method, an apparatus and/or a computer program product at any possible technical detail level of integration. The computer program product can include a computer readable storage medium (or media) having computer readable program instructions thereon for causing a processor to carry out aspects of the present invention. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer and/or computers, those skilled in the art will recognize that this disclosure also can be implemented in combination with other program modules. Generally, program modules include routines, programs, components, data structures, etc. that perform particular tasks and/or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A device, comprising:
a memory that stores computer executable components; and
a processor that executes the computer executable components stored in the memory, wherein the computer executable components comprise:
a training logic component that generates a prediction model, wherein the prediction model generates a predicted diagnosis based on electronic healthcare record data and image data of a known patient; and
a determination logic component that determines whether the predicted diagnosis exceeds an accuracy threshold value.

2. The device of claim 1, wherein responsive to a determination that the predicted diagnosis does not exceed the accuracy threshold value, the training logic component revises the prediction model based on the predicted diagnosis to produce a revised prediction model to diagnose an unknown medical condition of a new patient.

3. The device of claim 1, further comprising a correlation logic component that:
compares a known diagnosis with the predicted diagnosis; and
generates comparison results, wherein the determination logic component determines if the comparison results exceed the accuracy threshold value, and wherein responsive to a determination by the determination logic component that the comparison results do not exceed the accuracy threshold value, the training logic component revises the prediction model based on the comparison results to produce a revised prediction model, wherein the prediction model has a first level of accuracy in diagnosing an unknown medical condition of a new patient and the revised prediction model has a second level of accuracy of diagnosing the unknown medical condition of the new patient, and wherein the second level of accuracy is greater than the first level of accuracy.

4. The device of claim 2, further comprising:
an output logic component that outputs the revised prediction model for use by other medical devices to generate a new medical diagnosis based on new image data of the new patient.

5. The device of claim 1, wherein the prediction model identifies digestive track conditions.

6. The device of claim 1, wherein the image data indicates whether one or more of a group consisting of: polyps, bleeding, and an ulcer are associated with a lesion.

7. The device of claim 1, wherein the training logic component generates the prediction model employing a learning algorithm.

8. The device of claim 7, wherein the training logic component applies assisted learning techniques to the learning algorithm.

9. The device of claim 1, wherein the training logic component applies a directed learning algorithm to the prediction model.

10. The device of claim 1, wherein the electronic healthcare record data is comprised of different data collected at different times and medical consultation reports.

11. The device of claim 1, wherein the prediction model is employed to detect at least one of the group of: internal lesions, cancer, aneurisms, tumors, and arteriovasuclar malformations.

12. The device of claim 1, wherein the image data is selected from a group consisting of: a computed tomography image, an ultrasound image, and an X-ray image.

13. The device of claim 1, wherein the prediction model is employed to diagnose digestive track conditions.

14. The device of claim 13, wherein the image data is endoscopy image data.

15. A computer-implemented method, comprising:
generating, by a system operatively coupled to a processor, a prediction model, wherein the prediction model generates a predicted diagnosis based on electronic healthcare record data and image data of a known patient; and
determining, by the system, whether the predicted diagnosis exceeds an accuracy threshold value.

16. The computer-implemented method of claim 15, wherein responsive to the predicted diagnosis being determined to not exceed the accuracy threshold value, revising, by the system, the prediction model to generate a revised prediction model.

17. The computer-implemented method of claim 16, further comprising generating an updated prediction model based on the revised prediction model.

18. A computer program product that facilitates medical diagnosis, the computer program product comprising a computer readable storage medium having program instructions embodied therewith, the program instructions are executable by a processor to:
generate, by the processor, a prediction model, wherein the prediction model generates a predicted diagnosis based on electronic healthcare record data and image data of a known patient; and
determine, by the processor, whether the predicted diagnosis exceeds an accuracy threshold value.

19. The computer program product of claim 18, wherein the program instructions are further executable to cause the processor to:
compare a known diagnosis with the predicted diagnosis; and
generate comparison results, wherein responsive to a determination that the comparison results do not exceed the accuracy threshold value, the processor revises the prediction model based on the comparison results to produce a revised prediction model.

20. The computer program product of claim 19, wherein the prediction model has a first level of accuracy in diagnosing an unknown medical condition of a new patient and the revised prediction model has a second level of accuracy of diagnosing the unknown medical condition of the new patient, wherein the second level of accuracy is greater than the first level of accuracy.
